(19) United States
(12) Patent Application Publication
(10) Pub. No.: US 2006/0116423 A1
(43) Pub. Date: Jun. 1, 2006

(54) Methods and compositions for treatment of central nervous system injury with isothiocyanates

(76) Inventor: Pramod K. Dash, Houston, TX (US)

Correspondence Address: CONLEY ROSE, P.C., P. O. BOX 3267, HOUSTON, TX, 77253-3267, US

(21) Appl. No.: 11/252,260

(22) Filed: Oct. 17, 2005

Related U.S. Application Data
(60) Provisional application No. 60/619,653, filed on Oct. 18, 2004.
(60) Provisional application No. 60/638,691, filed on Dec. 22, 2004.

(51) Int. Cl. A61K 31/26

(52) U.S. Cl. 514/514

(57) ABSTRACT

Methods and compositions comprising isothiocyanates or derivatives or metabolites thereof, for attenuating or preventing central nervous system tissue damage, and/or improving cognitive function, are disclosed. Treatment of head trauma, spinal cord injury, stroke, aging, neurological diseases, and other insults to the central nervous system that compromise the blood-brain barrier, cause brain swelling, central nervous system cell death, or cognitive or motor dysfunction, by administering an isothiocyanate such as sulforaphane, allyl isothiocyanate, phenylethyl isothiocyanate, or a related compound, is also disclosed.

### CROSS-REFERENCE TO RELATED APPLICATIONS

This application claims the benefit under 35 U.S.C. §119(e) of U.S. Provisional Patent Application No. 60/638,691 filed Dec. 22, 2004 and U.S. Provisional Patent Application No. 60/619,653 filed Oct. 18, 2004, the disclosures of which are hereby incorporated herein by reference.

### STATEMENT REGARDING FEDERALLY SPONSORED RESEARCH OR DEVELOPMENT

Part of the work performed during the development of this invention utilized U.S. Government funds. Accordingly, the U.S. Government has a paid-up license in this invention and the right in limited circumstances to require the patent owner to license others on reasonable terms as provided for by the terms of Grant No. NS35457 and NS049160 awarded by the National Institutes of Health.

### BACKGROUND OF THE INVENTION

1. Field of the Invention

The present invention generally relates to compounds and methods involving isothiocyanates and their medical use for attenuating blood-brain barrier breakdown, brain edema, central nervous system tissue damage, and motor and cognitive improvements.

2. Description of Related Art

Brain trauma, spinal cord injury and stroke are serious health problems, with stroke being the third leading cause of death and disability in the United States. Studies have shown that multiple biochemical mechanisms including glutamate-mediated excitotoxicity, generation of reactive oxygen species, inflammation, calcium-activated proteolysis, and apoptosis contribute to the pathophysiology of these conditions^{3,38}. Based on existing evidence, there is a consensus opinion that combination treatments are likely to offer greater protection than single target therapies¹⁹. Tissue plasminogen activator (tPA) for stroke has been approved for use in thrombolitic strokes. TPA is effective when administered soon after the onset of stroke, however if given late, can have serious side effects such as hemorrhage⁴. Only methylpredinisolone administration is a FDA approved treatment for spinal cord injury and has been reported to improve motor recovery⁴⁷. Unfortunately, methylpredinisolone was not effective in decreasing pathology and behavioral deficits following brain trauma⁹. Insults to the central nervous system such as traumatic brain injury or stroke often cause death. Those who survive suffer from persistent cognitive and motor deficits. So far, none of the clinical trials for brain trauma have yielded positive results.

Traumatic brain injury in humans can produce numerous behavioral and cognitive dysfunctions including memory impairments, attention deficits, motor abnormalities, post-traumatic depression and amnesia¹⁷. Both retrograde (memory of events preceding the trauma) and anterograde (memory of post-traumatic events) amnesia are salient consequences of brain trauma. Retrograde amnesia is graded such that recent memories are lost more easily than remote memories. Anterograde amnesia can sometimes be ungraded and extensive depending on the severity of injury.

The primary insult initiates a cascade of cellular, biochemical and molecular changes, which in turn activate secondary responses^{30,34}. Secondary injuries are physiological events occurring within minutes, hours or days after the primary injury that lead to further damage of the nervous system. The effects of secondary brain damage are epitomized by the so called “talk and deteriorate” patients who are able to talk after their traumatic injury but ultimately die, clearly demonstrating that primary mechanical injury is not the sole determinant of outcome^{30,34}. Breakdown of the blood-brain barrier (BBB), brain swelling (or edema) which can lead to uncontrollable intracranial pressure, inflammation, and loss of brain cells are some of the secondary events that contribute to death, and cognitive or motor deficits. Currently, no effective treatment is available to attenuate the breakdown of BBB, brain swelling or lessen cognitive and motor deficits.

Blood-brain barrier vs. Peripheral barrier. The BBB consists of a brain endothelial cell layer and tight junctions between these cells (FIG. 1). The existence of the BBB has been recognized since the late nineteenth century, which was first demonstrated by the German microbiologist Goldman, a student of Paul Ehrlich. Electron microscopic studies have shown that the BBB is made up of endothelial cells present in capillaries that penetrate the brain. Ultrastructural studies have also shown that endothelial cells in the brain differ fundamentally from those in peripheral tissues in two ways: first, brain endothelial cells have very few endocytotic vesicles, limiting the entry of molecules into the brain through these cells (often referred to as transcellular pathway); second, the brain endothelial cells are joined to each other by tight junctions (or zonula occludens) that severely restrict passage of molecules including ions between endothelial cells (often referred to as paracellular pathway). Consistent with this, electrical resistance measurements have shown that brain capillaries have a unit resistance greater that 1,000-2,000 Ω/cm², compared to 10 Ω/cm² for peripheral capillaries². The high resistance tight junctions, and the paucity of endocytotic vesicles, are thought to develop through the interaction of endothelial cells with the brain tissue, possibly via secreted factors. Therefore, peripheral capillaries or cultured brain endothelial cells do not form the high resistance barrier and are fundamentally different than their in vivo counterparts.

For proper brain functioning, a tight control of permeability between blood and the brain is necessary. The molecular mechanism(s) underlying transcellular transport of molecules across the BBB is not well understood. Tight junction proteins allow for cell-to-cell adhesion thereby restricting movement of molecules via the paracellular pathway. Tight junction proteins are coupled to the actin cytoskeleton via specific intracellular proteins (e.g., ZO-1). The paracellular pathway is regulated by the opening of tight junctions and/or by rearrangement of actin cytoskeleton altering cellular morphology. Agents or insults can increase the BBB permeability by; a) causing death of endothelial cells, b) modifying junctional proteins (e.g., protein levels, proteolysis), c) causing rearrangement of actin cytoskeleton and cell retraction, d) causing dissociation of junction proteins from the actin cytoskeleton (e.g., via phosphorylation). For example, increases in intracellular cAMP leads to elevation of tight junction resistance and reorganization of actin cytoskeleton within the cells, and compounds such as lysophosphatidic acid (LPA) have been shown to increase BBB permeability^{35,36}.

There is little disagreement in the art as to whether brain trauma (or other pathological conditions) increases blood-brain barrier permeability, although the cause(s) underlying this have been debated. It is likely that a combination of mechanisms including the death of endothelial cells, increased endocytosis, altered expression of water channels, changes in junctional protein levels, proteolysis of junctional proteins, protein phosphorylation, actin cytoskeleton reorganization and/or endothelial cell retraction as a result of brain trauma contribute to increased BBB permeability.

It is thought that the loss of brain endothelial cells and or tight junctions as a result of insult (e.g., brain trauma, spinal cord injury, stroke) contributes to increased BBB permeability. Furthermore, infection (e.g., bacterial or viral meningitis) often results in increased BBB permeability. Compromised BBB leads to entry of blood product into the brain. This can result in brain swelling (or edema), production of free radicals and toxic molecules, and inflammation.

Edema. About 50% of patients with severe brain injury have some degree of brain edema. In about 25% of these cases, the edema occurs immediately after injury and is associated at autopsy with evidence of mortal injury to the brain. In the other three-quarters of patients, however, the process begins on the second or third day following injury and either progresses to untreatable elevated intracranial pressure or resolves by about the tenth day after injury. Damage to the brain from edema can be lessened if the intracranial pressures of the patients can be kept below 25 mm Hg. Current treatments aim to control intracranial pressure include: drainage of cerebrospinal fluid, hyperventilation to lower arterial CO₂ and constrict cerebral arteries, and administration of osmotic diuretics. Removal of the skull is even being done in some cases to relieve elevated pressures when other methods do not work. These treatments, however, all aim to reduce the impact of brain edema as there is no effective treatment to prevent its occurrence.

Brain edema, the infiltration and accumulation of excess fluid in the brain, which leads to an increase in brain tissue volume, is a key determinant of the morbidity and mortality following traumatic brain injury (TBI)^{8,23,29}. Two types of edema contribute to the overall increase in brain tissue volume. The first is vasogenic, in which water enters the brain as a result of the blood-brain barrier (BBB) compromise and accumulates in the extracellular space. Since the brain is confined within the skull, entry of fluid increases intracranial pressure. The second type of edema is cytotoxic, in which water enters cells causing them to swell¹³. The cellular and molecular mechanisms contributing to the development and resolution of TBI-associated brain edema are not well understood.

Aquaporin channels play an important role in water transport in many cell types. In the aquaporin water channel family, aquaporin-4 (AQP4) is the predominant subtype in the central nervous system (CNS) and is highly expressed in brain astrocytes, notably in the end-feet that surround brain capillaries^{28,32}. Recent studies have demonstrated that osmotic water flow through AQP4 is a mechanism that underlies cytotoxic brain edema^{21,22}. The expression of AQP4 is induced in the peri-infarcted tissue which is associated with the formation of the brain edema following focal cerebral ischemia⁴². Mice lacking aqp4 gene show significantly reduced brain edema and lethality in response to acute water intoxication or stroke²². These and other findings have led to the suggestion that water entry into cells through AQP4 may be detrimental under these conditions (water intoxication and cerebral ischemia) in which cytotoxic brain edema is predominant. However, it has also been proposed that AQP4 may function to clear excess water from the brain thereby decreasing vasogenic edema and intracranial pressure²². Consistent with this, direct infusion of isotonic fluid into the parenchyma caused a marked increase in brain water content and ICP in aqp4−/−mice as compared to wild type siblings²⁹.

Increases in free radicals and other toxic molecules are widely recognized as important to central nervous system pathophysiology¹⁰. Studies have reported increased production of reactive oxygen species such as the superoxide, hydroxyl radicals, peroxynitrite anion and other toxic molecules following trauma and stroke. If not detoxified, these molecules can react with and thereby functionally modify critical biomolecules including membrane lipids, proteins and DNA. Free radical scavengers when administered before or shortly after the primary insult are effective in lessening some of the secondary events in animal models. However, clinical trials for some of these scavengers have not yielded positive results, possibly because of their narrow range of action (i.e., designed for a specific type of radical) or due to the short half-life of these scavenging agents. Since injuries to the central nervous system are likely to increase many radicals and toxic molecules, a broadly acting agent is needed for effective therapy.

Isothiocyanates. Isothiocyanates are found in cruciferous vegetables, including broccoli, brussel sprouts, cabbage, and cauliflower. These compounds have gained attention because of their chemopreventive action in animals and human cell cultures^{40,44.} The specific effect of isothiocyanates seems to be dependent on the form of isothiocyanate, treatment regimen, and target tissue. Isothiocyanates are conjugated to glutathione in the body and excreted into the urine as their corresponding mercapturic acids²⁷. Therefore, mercapturic acids can be used as a biomarker for cruciferous vegetable intake⁴⁵.

The isothiocyanate sulforaphane [(−)-1-isothiocyanato-(4R)-(methylsulfinyl)butane], also referred to herein as SUL or SF, is a naturally occurring isothiocyanate that is present in abundance in cruciferous vegetables such as broccoli, has gained attention as a potential chemopreventative compound⁴⁸. SUL induces several classes of genes implicated in detoxifying reactive oxygen species and electrophiles (e.g., NADPH quinone oxidoreductase; NQO1, glutathione-S-transferases; GSTs), cell survival (e.g., insulin-like growth factor binding protein 2), calcium homeostasis (e.g. calcium-binding protein) and others^{12,44}. In vitro studies have shown that pretreatment of cortical cultures with SUL prior to hydrogen peroxide or glutamate exposure offers robust neuroprotection¹⁴, possibly through the combined action of the induced enzymes. The transcription factor NF-E2-related factor-2 (Nrf2), which binds to the antioxidant responsive element (ARE) with high affinity, is essential for the up-regulation of many of these genes following SUL exposure^{15,25}. Consistent with this, neural cells from Nrf2−/−mice are more vulnerable to oxidative stress, and this vulnerability is reduced when these cells are transduced with a functional Nrf2 construct^{14,16}.

Increases in glutathione levels have been reported to enhance the survival of cultured endothelial cells exposed to oxygen free radicals. There have been suggestions that sulforaphane and its derivatives may prevent neuronal degeneration. For example, U.S. Pat. No. 6,812,248 (Zhang et al.) describes administering certain compounds that elevate glutathione or at least one Phase II detoxification enzyme in diseased tissue. However, it is not known if glutathione can decrease blood-brain barrier permeability, brain edema and lessen cognitive dysfunction following brain trauma in animal models or in humans.

### SUMMARY OF THE INVENTION

The present invention provides broadly acting agents for use as prophylactic or intravenous (i.v.) medication to treat or prevent cognitive decline and/or damage to the blood-brain barrier, central nervous system and the neurovascular unit, and effective therapies employing such agents. Accordingly, therapeutic methods using one or more isothiocyanate compound, or a derivative or metabolite thereof, for lessening the effects of insults to the central nervous system are also disclosed herein. Such compounds include, but are not limited to, sulforaphane, allyl isothiocyanate and phenylethyl isothiocyanate. The term “damage” or “insult” to the central nervous system has its usual meaning in the art, including, but is not limited to, mechanical, chemical or functional injury to the brain or spinal cord such as trauma or stroke, shaken baby syndrome, diseases or infections of the central nervous system, and post-traumatic stress disorder in a mammal, especially a human. It is demonstrated herein that (1) sulforaphane or its derivatives can be used to attenuate blood-brain barrier permeability and brain swelling, (2) sulforaphane or its derivatives can decrease central nervous system cell death following injury, stroke or diseases, and (3) sulforaphane or its derivatives can lessen cognitive and/or motor deficits following central nervous system injury, stroke or diseases. It is also demonstrated herein that (4) post-injury administration of sulforaphane attenuated aquaporin-4 loss in the injury core and further increased aquaporin-4 level in the penumbra region; and (5) post-injury administration of sulforaphane reduced brain edema at 1 and 3 days following traumatic brain injury.

Sulforaphane is considered to be representative of isothiocyanate compounds, and related or derivative compounds (e.g., allyl isothiocyanate, phenylethyl isothiocyanate, and metabolites of isothiocyanate compounds), that have similar effects to those demonstrated herein for sulforaphane, allyl isothiocyanate or phenylethyl isothiocyanate. In this disclosure, where the context permits, references to “sulforaphane” or to “an isothiocyanate” are intended to include the chemical compound sulforaphane and related or derivative isothiocyanate compounds, for example, sulforaphane, allyl isothiocyanate, phenylethyl isothiocyanate, and metabolites of those compounds.

Accordingly, certain embodiments of the present invention provide a method of treating a mammal suffering from an insult to the central nervous system, or a portion thereof. As used herein, the term “mammal,” sometimes referred to herein as an “individual,” includes, but is not limited to human. The method comprises administering to the individual a pharmaceutically effective amount of at least one isothiocyanate, or a derivative or metabolite thereof, to provide at least one of the following results in the individual: deterring blood-brain barrier compromise (e.g., attenuate the breakdown of the blood-brain barrier); deterring loss of brain tight junction proteins; deterring central nervous system cell death; deterring brain swelling and/or intracranial pressure; and/or reducing brain infarct volume, thereby lessening or preventing detrimental effects of the insult to the central nervous system or portion thereof.

In certain embodiments, the method further provides at least one of the following effects in the individual from administering the isothiocyanate, or a derivative or metabolite thereof: enhancing the level of aquaporin-4 in brain astrocytes; enhancing expression of at least one ARE-driven gene, thereby attenuating brain swelling and/or intracranial pressure in the individual.

In certain embodiments of the method, the step of administering further provides at least one of the following effects: deterring behavioral dysfunctions in the individual; deterring cognitive dysfunctions in the individual, thereby further lessening detrimental effects of the insult to the central nervous system or portion thereof.

In certain embodiments of the method, the step of administering an amount of an isothiocyanate, a derivative, or metabolite thereof, is sufficient to lessen motor deficits in the individual arising from the insult. In certain embodiments of the method, the administering step comprises administering an amount of an isothiocyanate, or a derivative or metabolite thereof, sufficient to decrease loss of central nervous system tissue following the insult.

In certain embodiments of the method, the insult comprises trauma, stroke or disease. For example, traumatic brain injury or spinal cord injury. In certain embodiments in which the insult comprises stroke, the step of administering to the individual an effective amount of at least one isothiocyanate, or a derivative or metabolite thereof, decreases infarct volume in the brain of the individual.

In certain embodiments of the method, the disease comprises an infection of the central nervous system and the administering step comprises administering an amount of an isothiocyanate, or a derivative or metabolite thereof, to decrease intracranial pressure and/or cell loss following the infection.

In certain embodiments of the method, the administering step comprises administering to the individual a natural product, or a naturally derived product comprising an isothiocyanate, or a derivative or metabolite thereof. For example, one or more of sulforaphane, allyl isothiocyanate and phenylethyl isothiocyanate may be administered. In certain embodiments of the method, the administering step comprises administering an effective amount of an isothiocyanate, or a derivative or metabolite thereof, to overcome toxicity associated with a synthetic agent, wherein overcoming the toxicity decreases or prevents death of central nervous system cells in the individual after exposure to the synthetic agent, a derivative, or metabolite thereof, as a result of the insult to the central nervous system.

Also provided in accordance with certain embodiments of the present invention is a method of attenuating aquaporin-4 loss in injured brain tissue. This method comprises administering to an individual suffering from a brain injury an effective amount of an isothiocyanate, or a derivative or metabolite thereof, to enhance the levels of aquaporin-4 in at least one area of injured brain tissue in the individual. In some embodiments, the step of administering leads to attenuation of aquaporin-4 loss following brain injury. In some embodiments, the step of administering leads to enhancement of aquaporin-4 levels following brain injury.

Further provided in accordance with certain embodiments of the present invention is a method of attenuating working memory defects or improving memory in a mammal. This method comprises administering a pharmaceutically effective amount of an isothiocyanate, or a derivative or metabolite thereof, to an individual suffering from a working memory deficit, thereby improving working memory function in the mammal. A working memory deficit may arise, for example, from a brain injury, or it may arise due to aging of the individual.

Also provided in accordance with certain embodiments of the present invention is a method of improving memory extinction, as may occur in a brain injured mammal or due to aging of an individual. This method comprises administering a pharmaceutically effective amount of an isothiocyanate, or a derivative or metabolite thereof, to an individual suffering from an impaired ability to extinguish memory of a traumatic event, thereby improving memory extinction in the mammal.

Still further provided in accordance with certain embodiments of the invention is a method of improving memory in a mammal in need thereof This method comprises administering a pharmaceutically effective amount of an isothiocyanate, or a derivative or metabolite thereof, to a mammal suffering from memory impairment or deficiency, to improve memory in the mammal. The memory impairment or deficiency may be, for example, age-related impairment of declarative or spatial memory, or it may arise from a brain damage in the individual.

Also provided in accordance with certain embodiments of the present invention is a central nervous system tissue protective agent that comprises a pharmaceutically acceptable carrier and an amount of isothiocyanate, preferably sulforaphane, a derivative, or metabolite thereof, that is capable of attenuating central nervous system tissue damage when the agent is administered to in an individual in need thereof and suffering from an insult to the central nervous system. In some embodiments, the agent contains an amount of sulforaphane, allyl isothiocyanate, phenylethyl isothiocyanate, a derivative, or metabolite thereof, that is capable of decreasing blood-brain barrier permeability and/or brain edema when the agent is administered to an individual in need thereof and suffering from an insult to the central nervous system. In some embodiments, the agent contains an amount of sulforaphane, allyl isothiocyanate, phenylethyl isothiocyanate, a derivative, or metabolite thereof, that is capable of decreasing brain insult-associated cognitive deficits, including spatial and declarative memory deficits, when the agent is administered to an individual in need thereof and suffering from trauma to the central nervous system. In some embodiments, the agent contains an amount of sulforaphane, allyl isothiocyanate, phenylethyl isothiocyanate, a derivative, or metabolite thereof, that is active for decreasing brain insult-associated motor deficits when the agent is administered to an individual in need thereof and suffering from insult to the central nervous system.

In some embodiments, the agent contains an amount of sulforaphane, allyl isothiocyanate, phenylethyl isothiocyanate, or a derivative, or metabolite thereof, that is active for slowing or preventing cognitive decline in processes such as aging, and cognitive improvement, when the agent is administered to an individual in need thereof.

Advantageously, as an alternate to combination treatments in which a number of therapies are directed at respective biochemical mechanisms that contribute to pathophysiology, a single agent such as an isothiocyanate acting at multiple pathological targets is expected to provide similar or better protection to the patient. These and other embodiments, features and advantages of the present invention will be apparent with reference to the following description and drawings.

### BRIEF DESCRIPTION OF THE DRAWINGS

FIG. 1 is a conceptual drawing of a cross-section of a brain capillary and the endothelial cell layer. The inset shows the some of key proteins involved in tight junction formation.

FIG. 2 shows that intra-peritoneal administration of sulforaphane following brain trauma decreases blood-brain barrier opening (measured using Evans blue extravasation) over time as compared to vehicle treated rats.

FIG. 3 shows that oral administration of sulforaphane can decrease blood-brain barrier permeability.

FIG. 4 is a bar graph showing the comparative effects of post-injury administration of sulforaphane, allyl isothiocyanate and phenethylisothiocyanate on blood-brain barrier permeability.

FIGS. 5A-C is a photomicrograph of immunostained rat brain sections for endothelial barrier antigen (EBA) from a sham (FIG. 4A), an injured animal receiving vehicle (FIG. 4B) and an injured animal receiving sulforaphane (FIG. 4C).

FIGS. 6A-C is a photomicrograph of immunostained rat brain sections for rat endothelial cell antigen (RECA) from a sham (FIG. 6A), an injured animal receiving vehicle (FIG. 6B) and an injured animal receiving sulforaphane (FIG. 6C).

FIG. 7 a bar graph showing summary results for EBA and RECA immunostaining from sham, injured animals receiving vehicle and injured animal treated with sulforaphane.

FIG. 8 is a bar graph showing summary results for claudin, occludin, ZO-1 protein and von-Willebrand factor (vWF, an endothelial cell marker) levels from sham, injured animals receiving vehicle and injured animals treated with sulforaphane.

FIGS. 9A-G are photographs and a bar graph showing that post-injury sulforaphane administration attenuates the loss of AQP4 in the injury core. Low magnification confocal images demonstrating AQP4 immunoreactivity in the parietal cortex of (FIG. 9A) a sham, (FIG. 9B) an injured animal receiving vehicle, and (FIG. 9C) an injured animal receiving SUL at 24 hr following TBI. High-magnification double-label immunohistochemistry pictures showing AQP4 and vWF immunoreactivities in (FIG. 9D) a sham, (FIG. 9E) an injured animal receiving vehicle, and (FIG. 9F) an injured animal receiving SUL at 24 hr following TBI. (FIG. 9G) Summary data for fluorescence intensity for AQP4 showing that TBI markedly decreases AQP4 immunoreactivity in the injury core, which is attenuated by SUL.

FIGS. 10A-H are photographs and bar graphs showing that post-injury sulforaphane administration further enhances AQP4 immunoreactivity in the penumbra region. Representative confocal images of AQP4 immunoreactivity from a sham (FIGS. 10A, D), an injured animal receiving vehicle (FIGS. 10B, E) and an injured animal receiving SUL (FIGS. 10C, F) at 24 hr after injury. (FIG. 10G) Summary data show that AQP4 fluorescence intensity is augmented by SUL at both 24hr and 3 days following injury. (FIG. 10H) Summary data show that there is no difference of GFAP fluorescence intensity in the penumbra region between injury-vehicle and injury-SUL groups at 24 hr after injury. Data are presented as the mean±S.E.M. Sham n=8, Vehicle n=8, SUL n=8. *P<0.05. Scale bars represent 100 μm (FIGS. 10A, B and C) and 20 μm (FIGS. 10D, E and F).

FIGS. 11 is summary data showing that SUL increases AQP4 mRNA level measured at 24-hour post administration. Data are presented as the mean±S.E.M. *, P<0.05 (n=3 for each group).

FIG. 12 is a bar graph showing that early post-injury administration of sulforaphane decreases brain swelling at 24 hr following injury.

FIG. 13 is bar graph showing that early post-injury administration of sulforaphane decreases brain swelling at 3 days after injury.

FIG. 14 is a bar graph showing that delayed post-injury administration of sulforaphane decreases brain swelling at 3 days after injury.

FIGS. 15A-B are photographs and graphs showing that post-MCA/CCA occlusion administration of SUL decreases infarct. Representative pictures of 2 mm-thick TTC stained brain slices from a vehicle—(FIG. 15A) and a SUL-treated (FIG. 15B) animal. Healthy pink tissue appears dark gray and infracted tissue appears white in these images.

FIG. 16 is summary data showing the total infarct volume from vehicle-treated (n=8) and SUL-treated (n=7) groups. *, P<0.05.

FIG. 17 is a line graph demonstrating working memory by comparing the abilities of treated and untreated brain injured rats to find the hidden platform in a working memory version of a water maze task.

FIGS. 18A-B are graphs demonstrating memory extinction ability in rats subjected to foot-shock. FIG. 18A compares the effect of sulforaphane treatment on extinction training. FIG. 18B shows the memory for the extinction event, tested 48 hr following the extinction training.

FIG. 19 is a graph demonstrating spatial memory comparing the abilities of vehicle- and SUL-treated brain injured rats in the hidden platform version of a water maze task.

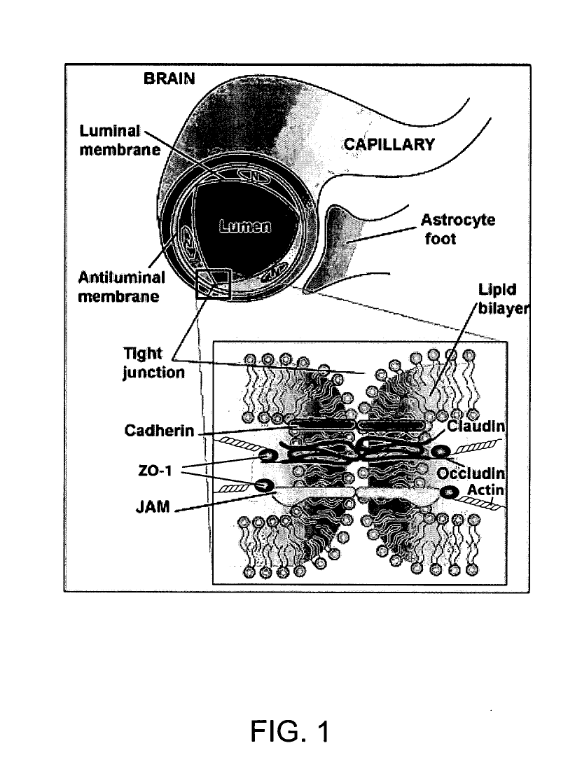

### DETAILED DESCRIPTION OF THE PREFERRED EMBODIMENTS

Several mechanisms are thought to contribute to the pathophysiology of brain damage. For example, traumatic brain injury alters BBB permeability, causes edema and impair cognitive functions. Both cell loss and/or altered cellular function are thought to underlie these pathologies. An agent that can alter multiple biochemical pathways may be more effective in decreasing brain damage than an agent acting on a single mechanism (e.g., preventing loss of endothelial cells). For example, although glutathione has been reported to enhance survival of cultured endothelial cells in vitro (cells that have been removed from the brain microenvironment) in response to oxygen free radicals, the death of brain endothelial cells in vivo is unlikely to be the sole contributor to brain damage. In the examples which follow, animal studies are presented which demonstrate that sulforaphane can decrease blood-brain barrier permeability, reduce brain edema and improve cognitive function following brain trauma. Since TBI primarily causes vasogenic edema as a result of compromised BBB, a decrease or loss of aquaporin-4 levels could delay the clearance of the excess water out of the brain. As described more fully in an example that follows, it was found that an isothiocyanate enhances aquaporin-4 expression and decreases cerebral edema following traumatic brain injury. In addition, SUL can decrease infarct volume following stroke in a clinically relevant animal model.

The following examples are offered by way of illustration, and not by way of limitation. Those skilled in the art will recognize that variations of the invention embodied in the examples can be made, especially in light of the teachings of the various references cited herein, the disclosures of which are hereby incorporated herein by reference.

General Methods and Materials.

Sulforaphane (99% Catalog No. S8044) for the studies disclosed herein was purchased from LKT Laboratories, Inc. St. Paul, Minn. USA. The drug was dissolved in corn oil and administered either orally or by intraperitonial (ip) injections. Anti-GFAP and anti-NeuN antibodies (Chemicon; Temecula, Calif.), and species-specific secondary antibodies conjugated to AlexaFluors (Molecular Probes, Carlsbad, Calif.) were purchased for this study. Male rats (Harland, Indianapolis, Ind.) weighing 350-375g were housed under temperature-controlled conditions with a 12-hour light/dark cycle and ad libitum access to water and food. Animal protocols were approved by the Institutional Animal Welfare Committee and were in compliance with NIH's Guidefor Care and Use of Laboratory Animals.

Cortical Impact Injury of Rats.

This model utilizes a pneumatic piston to deform a controllable volume of exposed cortex over a range of impact velocities. All protocols were in compliance with NIH's Guide for the Care and Use of Laboratory Animals and approved by the Institutional Animal Care and Use Committee. A controlled cortical impact device was used to cause brain trauma as previously described^{6,7,26}. Rats were anesthetized using 5% isoflurane with a 1:1 N₂O:O₂ mixture. The animals were then maintained with a 2% isoflurane/2:1 N₂O:O₂ mixture, via a face mask. Animals were mounted in the injury device stereotaxic frame secured by ear bars and an incisor bar. The head was held in a horizontal plane with respect to the interaural line. A midline incision was made, the soft tissues reflected, and bilateral 6 mm craniectomies were made midway between the bregma and lambda with the medial edge of each craniectomy 1 mm lateral to midline. Rats received a single impact at 1.8 mm deformation with an impact velocity of 6 m/sec at an angle of 20° from the vertical plane. Core body temperature was maintained at 37-38° C. by use of a rectal thermometer coupled to a heating pad. Sham rats were anesthetized and received craniectomies but were not cortically impacted. After injury or sham operation, the scalp was sutured and the animal placed in an oxygenated chamber and monitored during recovery. All rats were allowed to completely recover from the anesthesia after the surgery in a warm chamber before being sent back to the home cages. The type of brain trauma employed in this study produces both motor and cognitive deficits similar to those seen in human patients⁶.

Measurement of Blood-Brain Barrier (BBB) Integrity.

Blood-brain barrier integrity was assessed using extravagation of Evan's blue (EB) dye. When injected through the jugular vein of normal animals, EB is not detected in the nervous tissue. However, if the blood-brain barrier is compromised, EB can be detected in nervous tissues. The amount of EB can be quantified by extracting the dye from the tissue followed by spectrophotometric analysis at 610 nm wavelength. One and one-half hours prior to the above time points, animals were anesthetized and injected with 3% EB in saline (4 ml/kg) through the jugular vein. Animals were transcardially perfused with phosphate-buffered saline (PBS) until the out-coming liquid was clear followed by 4% paraformaldehyde (400 ml). The brain was removed and 2 mm-thick sections were prepared and incubated with 3 ml of 99.5% formamide in a 60° C. water bath for 24 hr. At the end of the incubation, tissues were removed and the formamide solution was centrifuged in an Eppendorf centrifuge at 12,000 rpm for 20 min. The supernatant solution was collected and the optical density at 610 nm was measured to determine the relative amount of EB dye in each sample.

Measurement of Brain Swelling (Edema).

Entry of blood fluid into the brain is thought to be a major contributor to brain swelling. Percent brain water content is determined using the wet and dry weights of brain tissue samples^{24,37,39}. At specific time points animals are killed by decapitation, brains are quickly removed and the cerebella discarded. Ipsi- and contralateral hemispheres are separated and each hemisphere is measured for tissue wet weight (WW). The tissues are then completely dried in a desiccating oven at 100-105° C. for at least two days until a stable dry weight (DW) is obtained. The percent water content (% H₂O) is calculated for each hemisphere as follows: % H₂O=[wet weight—dry weight)/wet weight]×100. The difference in percent water content between the ipsilateral (injured side) and contralateral (uninjured side) is compared to determine any effect of a drug on brain swelling.

Focal Ischemia.

Focal ischemia was induced by temporary left common carotid/middle cerebral artery (CCA/MCA) occlusion^{1,41}. Briefly, rats were anesthetized with chloral hydrate (0.45 g/kg IP). Core body temperature was maintained at 36.5±0.5° C. during ischemia The ipsilateral CCA was isolated and tagged through a ventral, cervical midline incision. A 0.005-inch-diameter stainless steel wire (Small Parts Inc) was placed underneath the left MCA rostral to the rhinal fissure, proximal to the major bifurcation of the MCA, and distal to the lenticulostriate arteries. The artery was then lifted, and the wire was rotated clockwise to ensure occlusion. The CCA was next occluded with an atraumatic aneurysm clip. Cerebral perfusion at the cortical surface, 3 mm distal to the locus of the MCA occlusion, was measured with a laser-Doppler flowmeter (LDF) (model BPM2, Vesamedic). Only those animals that displayed a cerebral perfusion reading of 10 ml/min/100 g tissue (approximately 12% to 15% of the initial value) on the LDF scale (expressing relative values of cerebral perfusion) were included in the study. After 180 minutes of MCA/CCA occlusion, reperfusion was established by reversing the occlusion procedure. After the indicated duration of reperfusion (depending on the study), animals were reanesthetized with chloral hydrate and had intracardiac perfusion of saline. Perfused isolated brains were transferred into ice-cold PBS for sectioning. With the use of a Jacobowitz brain slicer, 2-mm-thick coronal sections were cut before staining with 2% 2,3,5-triphenyltetrazolium chloride (TTC) in PBS for 30 minutes at room temperature for infarct volume discrimination. Stained sections were then transferred to 10% phosphate-buffered formalin for storage before infarct volume determination.

Infarct Volume Analysis.

Morphometric determination of infarct volume was obtained with the help of a computer-based image analyzer operated by Brain software (Drexel University). Infarcts produced by the present protocol are restricted to cortical tissue. The infarct volume (mm³) was calculated from the difference between the volume of contralateral cortex and the volume of the TTC-stained (nonischemic) portion of ipsilateral cortex of each rat. This indirect measure of infarct volume, based on the assumption that the volumes of the ipsilateral and contralateral cortex are the same before ischemia, corrects the total infarct volume for the edema component.

Immunohistochemistry.

Rats were killed and brains were removed quickly and dip-frozen in −80° C. isopentane for 20 sec. Slide-mounted, 10 μm-thick coronal sections were dried at room temperature for 1 hr and then fixed with methanol at −20° C. for 20 min. The sections were blocked in a PBS solution containing 5% goat serum and 0.25% Triton X-100 at room temperature for 1 hour, followed by a 48 hr incubation at 4° C. in primary antibodies (1.0 μg/ml) in blocking solution. Sections were washed in PBS, and incubated with AlexaFluor-conjugated secondary antibodies for 3 hr. Following washing in PBS, slides were coverslipped with Fluoromount G (Fisher Scientific) and immunoreactivity visualized using a Bio-Rad MRC 1024 confocal microscope. Double- and triple-label immunohistochemistry was performed essentially as described above with the addition of cell-type specific antibodies to the primary antibody incubation mixture followed by detection using appropriate secondary antibodies.

Fluorescence Intensity Quantification.

Fluorescence intensity was quantified essentially as described previously¹¹. Briefly, images of immunofluorescence were captured using a Bio-Rad MRC 1024 confocal microscope and Olympus BX 50WI camera. The parameters used for image acquisition (including laser power, iris size, brightness, offset, etc.) were pre-set to minimize the background and optimize the signal. These parameters were kept constant across all subsequent groups. A stack of pictures was generated for each section by scanning through the section at a step thickness of 0.80 μm along the z-axis. MetaMorph 6.1 software was used to determine the fluorescence intensity of the stack of pictures. Three non-overlapping regions (844 μm×633 μm) in the parietal cortex from each section and two sections from each animal were used for imaging. The fluorescence intensities of the three regions were averaged for each section, and the two sections were averaged for each animal. The sections containing the contusion core corresponded to 2.0 mm caudal to bregma level, whereas the sections representing the penumbra were taken from 1.0 mm caudal to bregma level.

Real-Time PCR.

Animals were killed and parietal cortical tissues were quickly dissected out and frozen in dry ice. The frozen tissue was homogenized in 1.5ml of TriZol (Invitrogen) per 100 mg tissue, followed by addition of chloroform (1:5) and incubation on ice for 20 min. The homogenate was centrifuged at 14,000×g for 30 min, and total RNA was precipitated by isopropanol. 1.0 jig total RNA was reverse transcribed for 2 hours at 37° C. in a 20μl mixture containing 50 mM Tris-HCl pH 8.3, 75 mM KCl, 3.0 mM MgCl₂, 10 mM DTT, 2.5 μM random hexamer, 1.0 mM each dNTP, 20 U RNasin, and 200 U Superscript II reverse transcriptase. The level of expression of each target gene was quantified using a BioRad iCycler real-time PCR system (BioRad, Hercules, CA). Reactions were carried out in triplicate. Each 30 μl reaction mixture consisted of 1.5 μl of the cDNA and 18 mM Tris-HCl pH 8.3, 55 mM KCl, 2.0 mM MgCl₂, 0.2 mM of each dNTP, 0.5μM of each primer, 10 nM fluorescein, 1:75,000 dilution of Sybr green I, and 2 U AmpliTaq DNA polymerase. The following primer pairs were utilized for target mRNA amplification: for AQP4, forward: 5′-CCAGCTGTGATTCCAAACGGA-3′, reverse: 5′-GCACAGCGCCTATGATTGGTC-3′; for β-actin, forward 5′-CCCCATTGAACACGGCATT-3′, reverse: 5′-CATCTTTTCACGGTTGGCCTTA-3′. The amplification protocol consisted of 1 cycle at 95° C. for 3 min followed by 40 cycles at 95° C. for 30 sec, 58° C. for 30 sec, then 72° C. for 30 protocol, performed at the end of the amplification, consisted of 80 cycles beginning at 55° C. for 10 sec, after which the temperature was increased by 0.5° C./cycle. A standard curve for each target gene was generated to determine the linear range and amplification efficiency of each sample. The threshold cycle of each sample was fitted to the standard curve to calculate the relative copy number of the initial cDNA. The resultant data was analyzed using the iCycler iQ Real-Time Detection System software³³.

Extinction of Fear Memory.

On day 9 post-injury, animals were trained in a delay conditioning task. Following a 2 min period in which the animal was allowed to familiarize itself with the training chamber, a 10 sec tone (CS) was presented. During the last 2 sec of tone presentation, a 0.8 mA footshock (US) was delivered which coterminated with the tone. This procedure was repeated three times. During conditioning, rats quickly learn that the sound of the tone predicts the associated foot shock. The presentation of the tone at a later time point elicits a fear response (lack for movement, increased heart rate, stress hormone release, increase blood pressure, etc). Two weeks later, rats were given 9 trials of extinction training in a novel context in which the tone was presented as described above in the absence of the footshock. During each of the extinction trials, fear is monitored by recording freezing behavior in 2 sec increments. With continued presentations of the tone, rats displayed less freezing with each successive tone presentation, indicating extinction of the fear response. Forty-eight hours following the extinction training, memory for the extinction event was tested by placing the animal in a novel environment and recording freezing behavior in response to tone presentations.

Spatial Memory Assessment.

Declarative memory formation and storage is dependent on the function of the hippocampus. In rodents, hippocampal-dependent memory (spatial memory) can be assessed using the hidden platform version of the water maze task as described by us previously. On day 14 post-injury, spatial memory testing was initiated. In this task, rats are trained to find the location of a submerged platform (the location remains constant throughout the experiment) by giving the animal 4 training trials per day for 5 consecutive days. Each trial begins from a novel position in the tank, with the animal facing the wall. The room contains numerous extramaze cues which remain constant throughout the experiment. Each trial is separated by a 4 minute intertrial interval. The time required to find the hidden platform (recorded in seconds) is averaged for each of the 4 daily trials. The function of the hippocampus (and related structures in the temporal lobe) can be determined by monitoring the rate of latency reduction over the five day testing period.

Working Memory Assessment.

Traumatic brain injury in rodents causes working memory deficits that last for weeks, which can be evaluated using a modified version of the Morris water maze task (delay match-to-place task) as described by us previously. On day 21 post-injury, animals were given four trials in the working memory version of the Morris water maze task. Each testing session consisted of a location trial, a 5 sec delay, and a match trial. Each location trial was started from a random location with the hidden platform in a novel position. Once the platform was found, the animal was allowed to rest on it for 10 sec. The animal was then removed from the hidden platform for a 5 second delay period (and placed in the holding area). Following the delay, the animal was placed back into the water maze and allowed to search for the platform (the match trial). If the animal failed to locate the platform within 60 sec on any given trial, it was led there by the investigator. After each testing session, the animal is given a 4-minute intertrial interval (iti) before the next testing session with a new platform location. If an animal has intact working memory, the information on platform position learned during the location trial will help it find the platform faster during the match trial. If an animal has impaired working memory, it will find the hidden platform by chance during both the location and match trials. Thus, the decrease in latency between the location trials and the match trials is used as a measure of working memory.

Statistical Analysis.

Student's t-test for unpaired variables was used for evaluating the mRNA levels, immunofluorescent intensity, brain edema and total infarct volume. Two way analysis of variance (two-way ANOVA) was used for evaluating cognitive functions and time course for blood-brain barrier permeability. Results were considered significant at P<0.05. Data are presented as the mean±standard error of the mean (S.E.M.).

### EXAMPLE 1

### Attenuation of Blood-Brain Barrier Break-down from Brain Injury following Intraperitoneal Administration of Sulforaphane

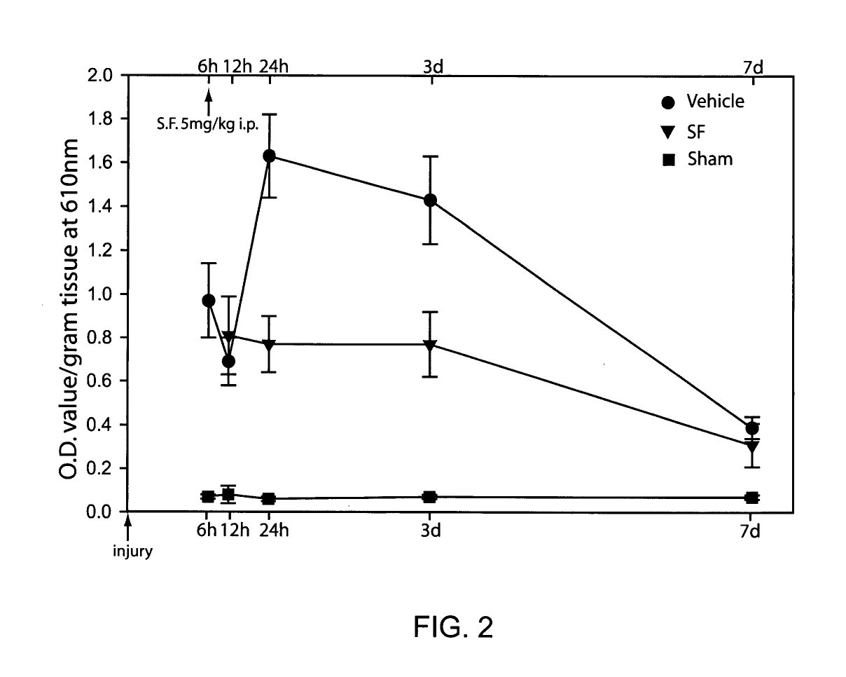

Six hours following injury or sham surgery, animals were administered with either 5 mg/kg of sulforaphane or equal volume of vehicle intraperitoneally (ip)⁴³. Blood brain barrier integrity was examined at 6 hr, 12 hr, 24 hr, 3 days and 1 week following injury by Evan's blue dye extravasation into the brain tissue. FIG. 2 shows the amount of dye in brain tissues (indicated by OD at 610 nm) for sham, injured animals receiving vehicle and injured animals receiving sulforaphane. The figure shows that an initial increase in the amount of dye in the brain tissue occurs by 6 hr following injury as compared to the sham group. This increase in dye extravasation is likely related to the primary insult. An additional, secondary increase in dye extravasation is detected at the 24 hr and 72 hr time points. Sulforaphane can significantly attenuate dye extravasation at these later time points. In FIG. 2, the time course for BBB permeability (ipsilateral side) is shown. Sulforaphane (SF) 5 mg/kg was given i.p. at 6 h after TBI. *P<0.05 (SF vs. Vehicle)

### EXAMPLE 2

### Attenuation of Blood-Brain Barrier Permeability by Oral Administration of Sulforaphane

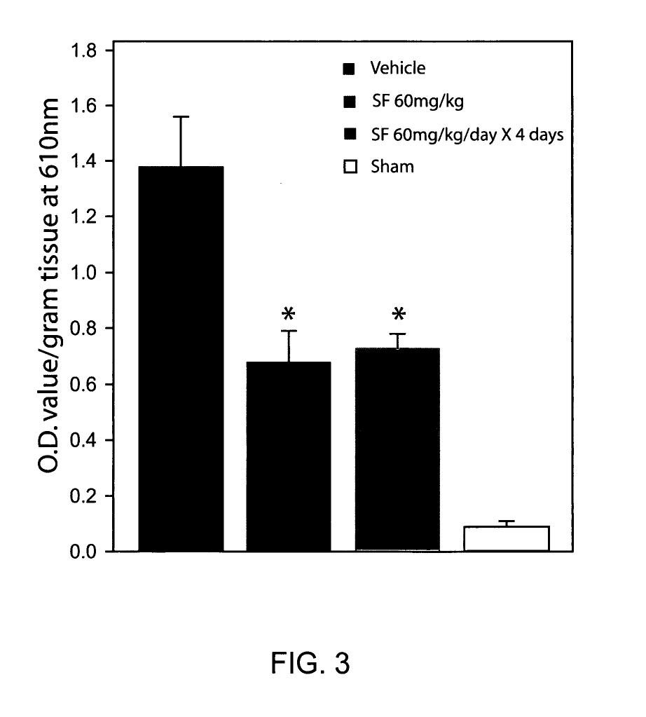

FIG. 3 is a bar graph showing that oral administration of sulforaphane (SF) (60 mg/kg/day for one or four days) is effective in decreasing the extravasation of Evan's Blue dye. Day one administration was given 6 hr following brain injury. *P<0.05 (SF vs. Vehicle)

### EXAMPLE 3

### Post-Injury Administration of Sulforaphane, Allyl Isothiocyanates and Phenylethyl Isothiocyanates Decrease Blood-brain Barrier Permeability.

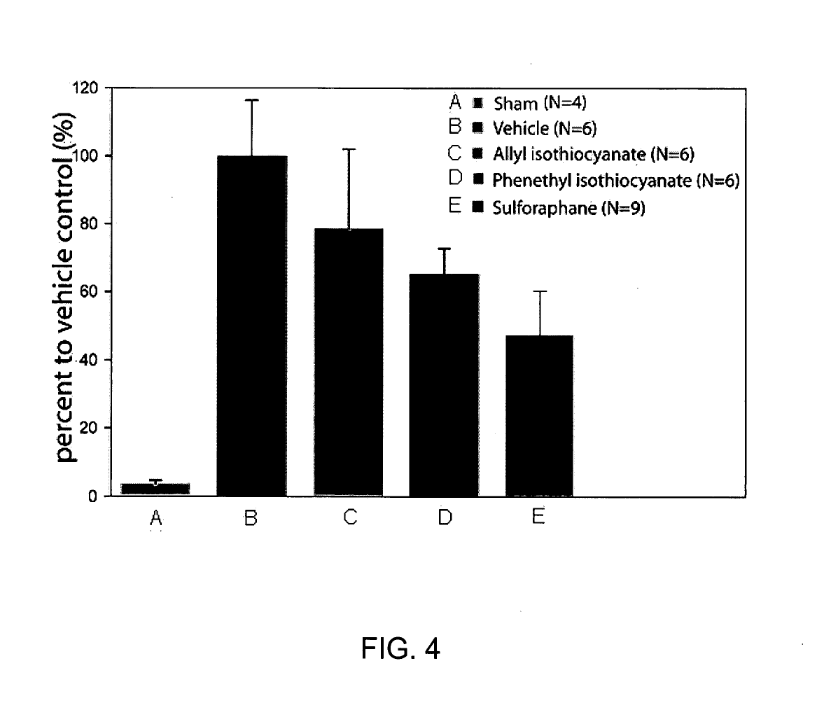

5 mg/kg sulforaphane, 2.8 mg/kg of allyl isothiocyanate or 4.6 mg/kg of allyl isothiocyanate (molar equivalents to 5 mg/ml sulforaphane) were administered i.p. 6 hr after brain trauma. Blood-brain barrier integrity following isothiocyanate administration was measured by Evans Blue extravasation into the brain 24 h after TBI brain trauma and compared to injured animals receiving vehicle. The results of those tests show that post-injury administration of each of the representative isothiocyanates sulforaphane, allyl isothiocyanate and phenylethyl isothiocyanate decreased blood-brain barrier permeability in the test model (FIG. 4).

### EXAMPLE 4

### Rescue of Brain Endothelial Cells and Tight Junction Proteins by Sulforaphane

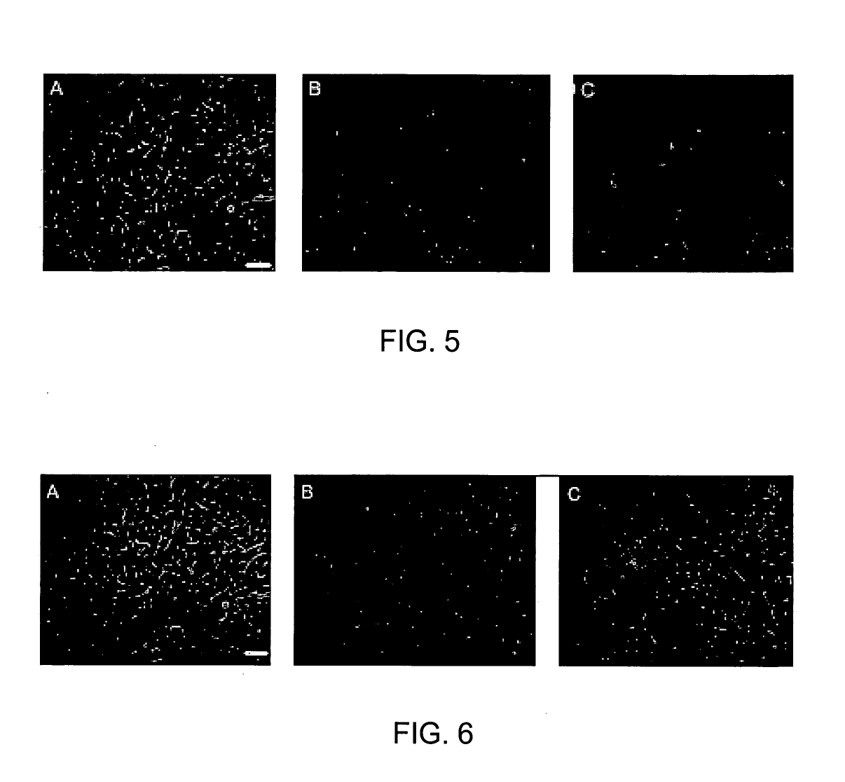

Brain endothelial cells and the tight junctions between them constitute a major component of the blood-brain barrier. Two antibodies: one for the endothelial barrier antigen (EBA) and the second for the rat endothelial cell antigen (RECA) were used to immunologically examine the protective effect of sulforaphane. In FIG. 5 representative pictures from brain sections showing sulforaphane treatment administered 6 hr after injury improves immunostaining for EBA are shown. Animals were killed 24 hr following injury. The scale bar is 100 μm. In FIG. 6 representative pictures from brain sections showing sulforaphane treatment administered 6 hr after injury improves immunostaining for RECA are shown. Animals were killed 24 hr following injury. The scale bar is 100 μm.

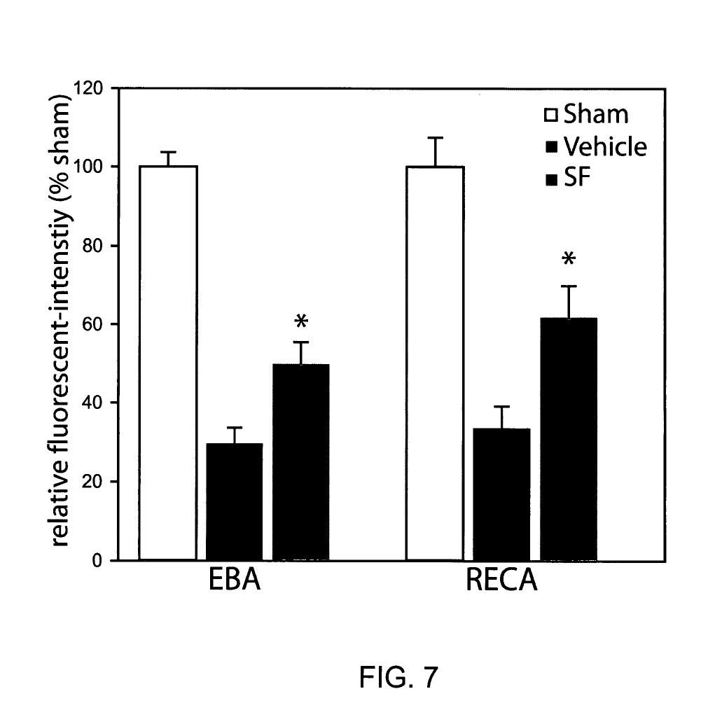

In order to assess if the increases in immunostaining for EBA and RECA shown in FIGS. 5 and 6 were significant following sulforaphane treatment, integrated fluorescent intensity (minus the background fluorescent intensity) was measured. FIG. 7 shows summary results indicating that sulforaphane treatment significantly improves EBA and RECA immunoreactivity. These findings suggest that the drug attenuates the loss of endothelial cells and barrier junctions. In FIG. 7, summary data for integrated fluorescent intensities for EBA and RECA staining from cortices ipsilateral to injury are shown. Sulforaphane (SF) significantly improves both EBA and RECA staining as compared to injured animals receiving vehicle. Sulforaphane was given i.p. at 6 h after injury and samples were collected at 24 h after injury. *P<0.05 (SF vs. Vehicle). n=7 for vehicle, SF 20 mg/kg n=5 and SF 5 mg/kg n=7, sham=2.

### EXAMPLE 5

### Delayed Administration of Sulforaphane Attenuates the Loss of Brain Tight Junction Proteins and an Endothelial Cell Marker

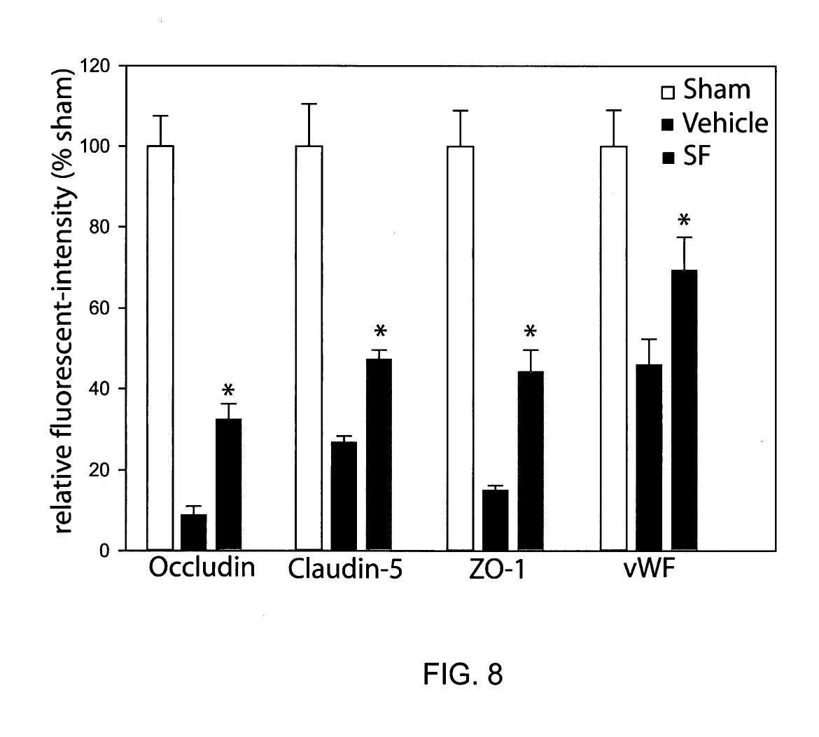

The effect of sulforaphane on brain tight junction proteins and endothelial cells following brain trauma was assessed by immunostaining brain sections with antibodies for claudin-5, occludin, ZO-1 and von-Willebrand factor (vWF) (an endothelial cell marker). Immunoreactivity was detected using fluorescently labeled secondary antibodies and quantified by laser confocal microscopy. FIG. 8 is a bar graph of the summary data showing that delayed administration of sulforaphane partially blocks loss of tight junction proteins and vWF. * P<0.05 (injury+vehicle vs. injury+sulforaphane). FIG. 8 shows that brain trauma causes drastic reductions in claudin-5, occludin, ZO-1 and von-Willebrand factor levels. Administration of sulforaphane six hours after injury significantly attenuated the loss of tight junction protein levels. In addition, injury-induced loss of vWF levels was partially restored by sulforaphane, a finding consistent with RECA staining in Example 3.

### EXAMPLE 6

### Sulforaphane Enhances Aquaporin-4 Expression following Traumatic Brain Injury

Using a rodent injury model, it is shown that TBI decreased aquaporin-4 (AQP4) level in the injury core and modestly increased AQP4 in the penumbra region surrounding the core. Post-injury administration of sulforaphane attenuated AQP4 loss in the injury core and further increased AQP4 levels in the penumbra regions as compared to injured animals receiving vehicle. These increases in AQP4 levels were accompanied by a significant reduction in brain edema (assessed by percent water content) at 3 days post-injury.

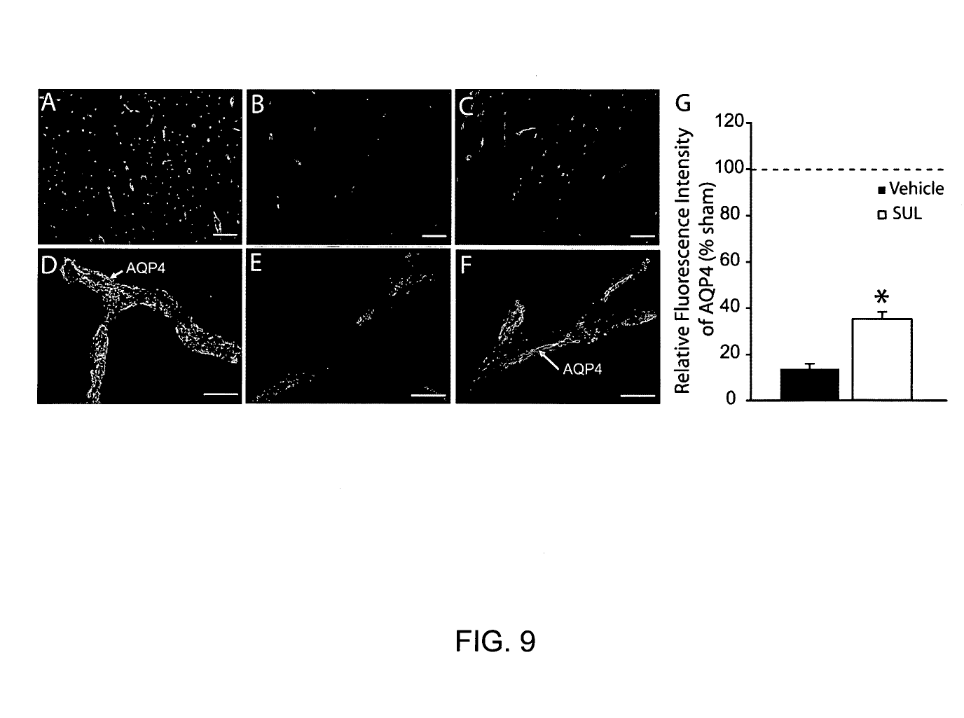

Sulforaphane attenuates the loss of AQP4 immunoreactivity in the injury core. To determine the influence of SUL on TBI-associated loss of AQP4 immmunoreactivity in the injury core, groups of injured animals were intraperitoneally (i.p.) injected with either SUL (5 mg/kg prepared in corn oil) or equal volume of vehicle at 6 hr after injury. AQP4 immunoreactivity was visualized using confocal microscopy. The representative low magnification pictures show that the marked reduction in AQP4 immunoreactivity in the contusion core at 24 hr after injury (sham animal, FIG. 9A vs vehicle-treated animal, FIG. 9B) was lessened as a result of SUL treatment (FIG. 9C). Double-label immunohistochemistry of representative brain capillaries (indicated by vWF immunoreactivity) shows that AQP4 immunoreactivity was dramatically decreased along vessels in the contusion core (FIG. 9E) compared to sham animals (FIG. 9D). By comparison to the vehicle control, the representative blood vessel from a SUL treated animal shows increased immunoreactivity for AQP4 (FIG. 9F). The quantification of AQP4 immuno-fluorescence intensity shown in FIG. 9G indicates that post-injury SUL administration modestly but significantly attenuated the loss of AQP4 immunoreactivity caused by the injury (24hr injury-vehicle:13.79±2.22%, 24 hr injury-SUL: 35.25±3.13%, P<0.05). Data is presented as the mean±S.E.M. Sham n=4, Vehicle n=4, SUL n=4. *P<0.05. Scale bars represent 100 μm (FIGS. 9A, B and C) and 20 μm (FIGS. 9D, E and F).

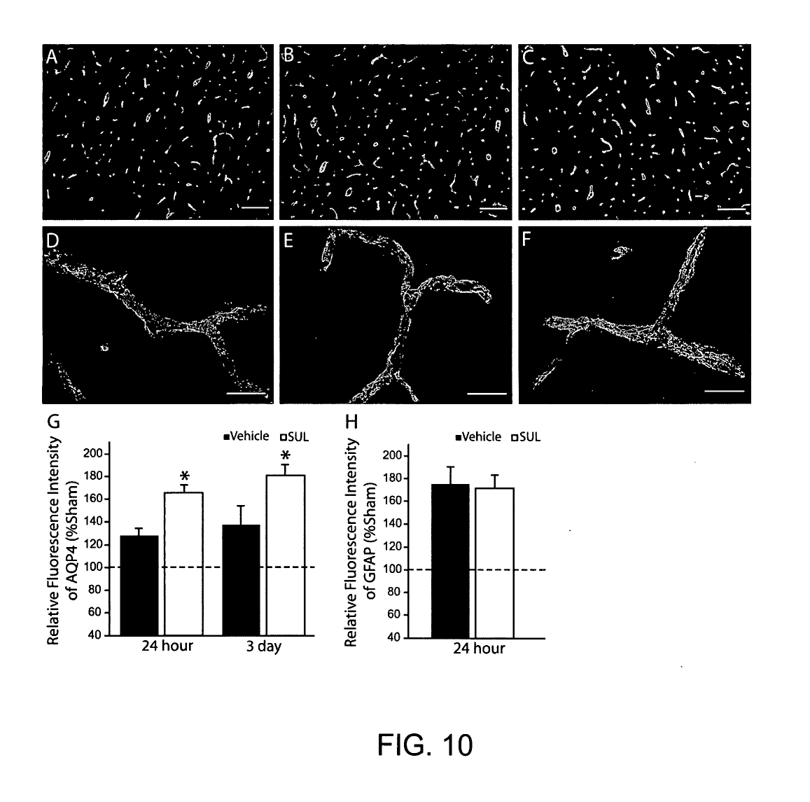

Sulforaphane increases AQP4 immunoreactivity in the penumbra region. Referring now to FIGS. 10A-F, representative confocal images are shown of AQP4 immunoreactivity from a sham (FIGS. 10A, D), an injured animal receiving vehicle (FIGS. 10B, E) and an injured animal receiving SUL (FIG. 10C, F) at 24 hr after injury. In FIG. 10G, summary data for AQP4 fluorescence intensity is shown. In FIG. 10H, summary data for glial fibrilary acidic protein (GFAP) fluorescence intensity in the penumbra region is shown. Data are presented as the mean±S.E.M. Sham n=8, Vehicle n=8, SUL n=8. *P<0.05. Scale bars represent 100 μm (FIGS. 10A, B and C) and 20 μm (FIGS. 10 D, E and F). In the penumbra region surrounding the contusion core, TBI modestly increased AQP4 immunoreactivity (FIGS. 10B and 10E) as compared to sham controls (FIG. 10A and 10D). Post-injury SUL administration further augmented AQP4 levels (FIGS. 10C and 10F). Summary results for AQP4 fluorescence intensity measurements (FIG. 10G) show that post-injury administration of SUL significantly increased AQP4 levels in the penumbra region at both 24 hr and 3 days following injury. The increase in AQP4 does not appear to be due to the enhancement in the number or the activation of astrocytes following injury as indicated by a lack of difference in GFAP immunoreactivity between vehicle and SUL-treated animals (FIG. 10H). Accordingly, it is suggested that SUL may increase the expression of AQP4.

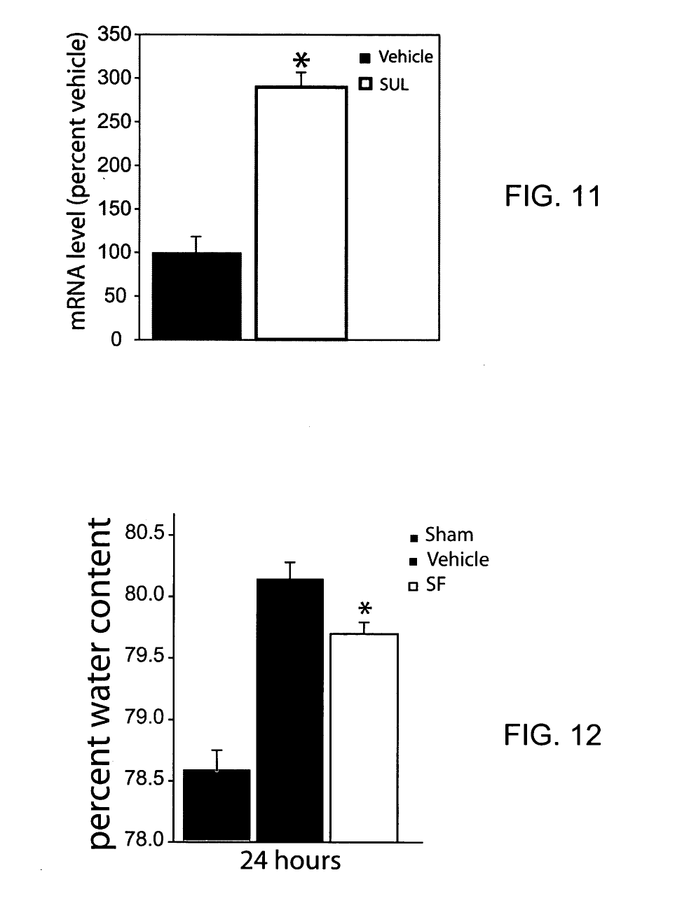

Systemic administration of sulforaphane increases AQP4 mRNA levels in the brain. To assess the ability of SUL to induce AQP4 expression, rats were intraperitonealy injected with either 5 mg/kg SUL prepared in corn oil or equal volume of vehicle. Twenty-four hours following SUL or vehicle administration, animals were killed and cortical tissues were dissected for RNA extraction. AQP4 mRNA levels were evaluated using real-time PCR. The summary data (from three independent experiments) in FIG. 11 shows that SUL administration significantly increases AQP4 mRNA level at the 24 hr post-administration time point (vehicle: 100.00±18.16%; SUL: 289.76±16.52%, P<0.05). The levels of β-actin mRNA were used as an internal control to evaluate the amount of starting material for each sample. No significant difference in β-actin mRNA level was observed in any of the samples. Data are presented as the mean±S.E.M. *, P<0.05 (n=3 for each group).

### EXAMPLE 7

### Early Administration of Sulforaphane Decreases Brain Swelling Following Injury

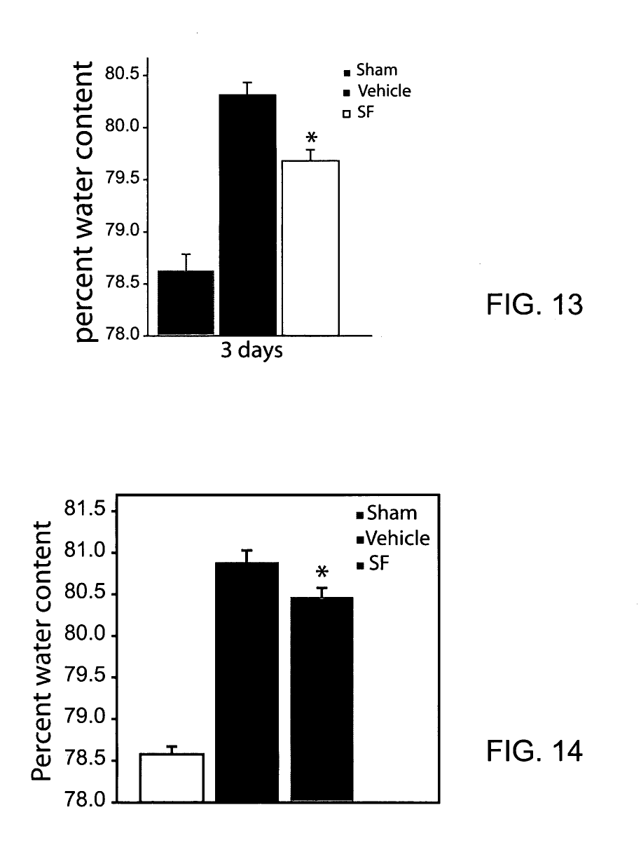

The effect of sulforaphane treatment on brain swelling (or edema) was measured by determining the percent water content at 24 hr and 3 day following injury. Referring now to FIG. 12, it is shown that sulforaphane (SF) decreases brain water content measured 24 hr following injury when given (5 mg/kg ip) at 15 min and at 6 hr following injury. Brain swelling was measured by the difference in percent water content between the injured and uninjured hemispheres. *P<0.05 (SF vs. Vehicle). With reference to FIG. 13, sulforaphane (SF) administration (5 mg/kg ip) given at 15 min and at 6 hr after injury significantly decreases the difference in brain water content measured at 3 day following the injury. Brain swelling was measured by the difference in percent water content between the injured and uninjured hemispheres. *P<0.05 (SF vs. Vehicle).

### EXAMPLE 8

### Decrease in Brain Swelling by Delayed Administration of Sulforaphane

It has been noted that it takes approximately 6 hr for a trauma patient to arrive at an emergency room and be stabilized for other injuries before a treatment for brain injury can be administered⁵. In order to examine if sulforaphane would be effective in rats if the administration is delayed, injured animals were given the drug (5 mg/kg ip) at 6 hr after injury. Animals were killed 3 days later and the percent water content was measured. Referring to FIG. 14, delayed administration of sulforaphane decreases brain swelling is demonstrated. Brain swelling was measured by the difference in percent water content between the injured and uninjured hemispheres. Animals were given the drug (5 mg/kg ip) at 6 hr following injury and water content was measured at 3 days after injury. *P<0.05 (SF vs. Vehicle). The results presented in FIG. 14 indicate that delayed administration of sulforaphane significantly reduces the increase in brain water content at the 3 day time point.

### EXAMPLE 9

### Sulforaphane Reduces Infarct Volume following Focal Cerebral Ischemia in Rodents

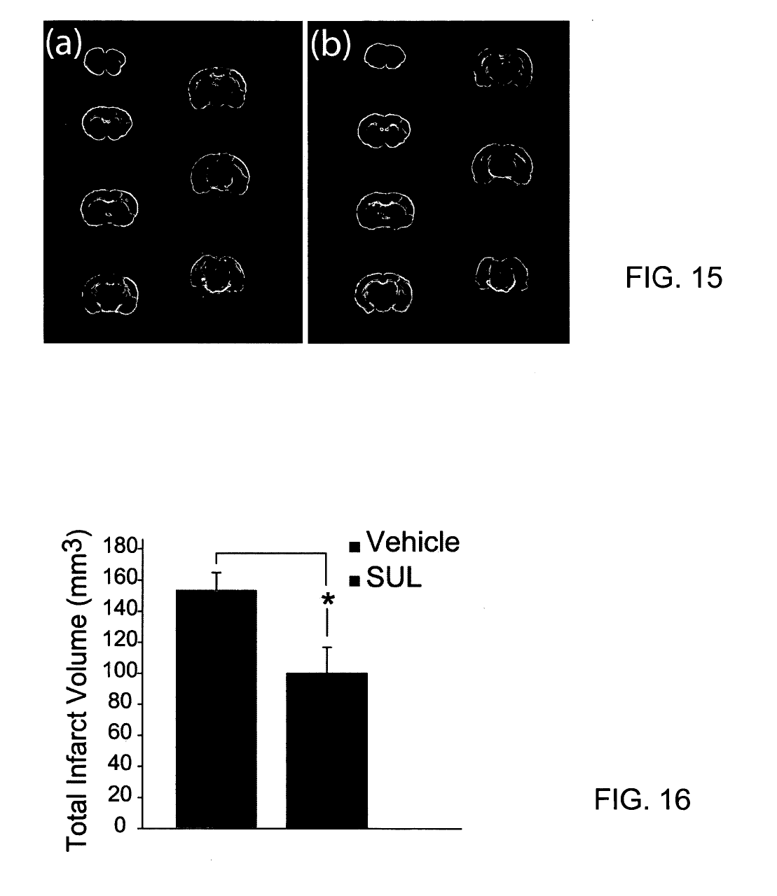

Further studies were carried out to determine if post-ischemia administration of SUL can reduce infarct volume resulting from common carotid/middle cerebral artery (CCA/MCA) occlusion. SUL at 5 mg/kg was dissolved in corn oil and injected intra-peritoneally (i.p.), 15 minutes after the onset of ischemia. Animals in vehicle group receive an equal volume of corn oil. After 3 hours of CCA/MCA occlusion, reperfusion was established by reversing the occlusion procedure. Three days following ischemia, infarct volume (mm³) was calculated from the difference between the volume of contralateral cortex and the volume of the TTC-stained (non-ischemic) portion of ipsilateral cortex of each rat. FIG. 15 shows representative series of 2 mm-thick brain sections from a vehicle—(FIG. 15A) and a SUL-treated (FIG. 15B) animal. Healthy tissue appears pink in these images. An apparent decrease in the size of the infarct (white tissue) can be seen in the images from SUL-treated animal. For quantification, the infarct volume was calculated using a computer-based image analyzer. The summary results (FIG. 16) indicate that SUL significantly reduced the total infarct volume (vehicle: 153.87±11.01 mm³, n=7; SUL: 100.52±16.35 mm³; n=8, P<0.05).

### EXAMPLE 10

### Sulforaphane Improves Working Memory in Brain Injured Rodents

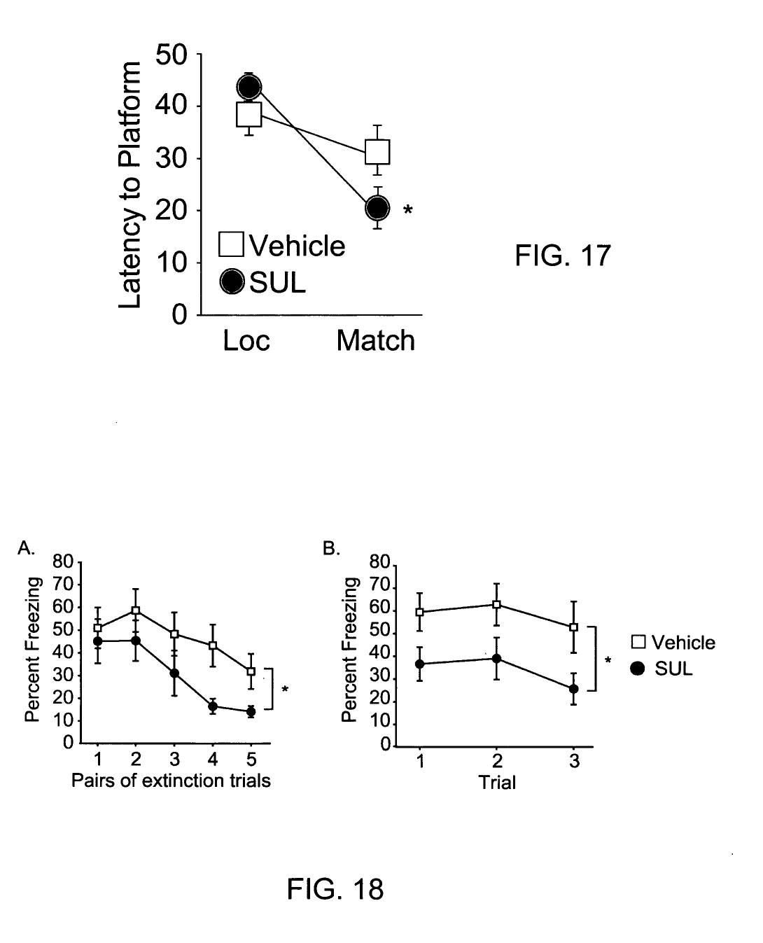

The prefrontal cortex (PFC) is highly vulnerable to traumatic brain injury (TBI). Most brain trauma patients report some cognitive deficits attributable to their PFC dysfunction, including problems with working memory and post-traumatic stress disorder (PTSD). Working memory involves holding information in mind that is necessary for performing both simple and complex cognitive operations. For example, remembering a phone number for the duration of dialing, or recalling the presence of a car when merging into traffic requires working memory. Working memory is also believed to make important contributions to higher cognitive functions such as reasoning, comprehension, planning, and spatial processing. Recently, the persistence of working memory deficits was demonstrated in brain-injured children, who still showed deficits 5 years after their injury¹⁸. To test if post-injury administration of sulforaphane improves working memory function, a group of injured animals was i.p. injected 6 hr post-injury with 5mg/kg sulforaphane. A second group, which was injected with vehicle, served as a control. Working memory testing was carried out on day 21 post-injury. FIG. 17 shows that injured rats treated with vehicle (n=8) have working memory impairments as indicated by a lack of a significant decrease in latency during the match trial as compared to the location trial. Sulforaphane-treated animals (n=8), by comparison, find the hidden platform significantly faster during the match trial, indicating improved working memory. *P<0.05 (two-way ANOVA).

### EXAMPLE 11

### Sulforaphane Improves Memory Extinction in Brain Injured Rodents

Post-traumatic stress disorder is thought to arise from an inability to forget (or extinguish) the memory of a traumatic event. As seen in humans with post-traumatic stress disorder, rats with frontal cortex damage (specifically the medial prefrontal cortex) have difficulty in remembering that a fear response has been extinguished. To test if post-injury administration of sulforaphane improves memory extinction, a group of injured animals was i.p. injected 6 hr post-injury with 5 mg/kg sulforaphane. A second group, which was injected with vehicle, served as a control. Animals were trained in the delay conditioning task on day 9 post-injury. Memory extinction was carried out on day 23 post-injury. Extinction was more rapid in the rats receiving sulforaphane (n=8) than those receiving vehicle (n=8) (FIG. 18A). *P<0.05 (two-way ANOVA). As extinction of fear memory is thought to depend on the integrity of the prefrontal cortex, the enhanced extinction in the sulforaphane group suggests improved prefrontal function. In addition, memory for extinction has been demonstrated to require prefrontal function. To assess if injured animals receiving sulforaphane also have enhanced memory of the extinction event, fear behavior was tested 48 hr following the extinction training. FIG. 18B shows that when freezing behavior was monitored during 3 tone presentations, vehicle-treated animals returned to pre-extinction freezing values, indicating that they forgot that the tone was not predictive of the foot shock. Sulforaphane-treated animals, by comparison, had a reduced freezing to the tone, suggesting that these animals remembered the extinction training in which they learned that the tone does not predict the foot shock. *P<0.05 (two-way ANOVA). Thus, the capacity to forget a traumatic memory is improved as a result of sulforaphane treatment of injured animals.

### EXAMPLE 12

### Sulforaphane Improves Spatial Memory in Brain Injured Rodents

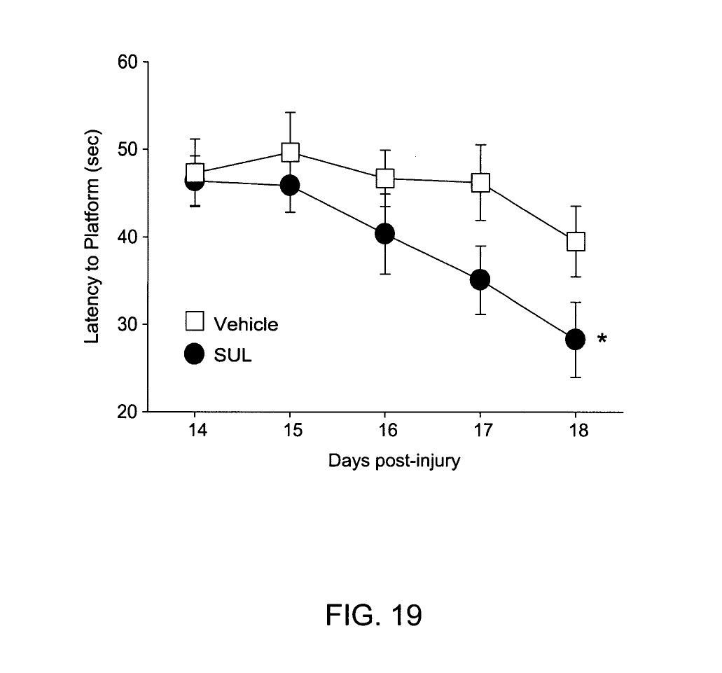

To test if spatial memory is improved in brain injured animals treated with SUL, rats were administered 6 hr post-injury with 5 mg/kg sulforaphane (n=9) or an equal volume of vehicle (n=9). On days 14-18 post-injury, rats were tested in the water maze task. Referring to FIG. 19, rats treated with SUL demonstrated improved spatial learning and memory as indicated by a more rapid reduction in latency to find the hidden platform compared to vehicle-treated animals. *P<0.05 (two-way ANOVA).

Discussion.

Several important conclusions were derived from these studies. First, there are two phases to the blood-brain barrier permeability following brain injury. The delayed component can be significantly attenuated both by oral and i.p. administration of an isothiocyanate. Second, loss of EBA and RECA immunoreactivity as a result of injury can be attenuated by an isothiocyanate, indicating that an isothiocyanate can protect against the loss of endothelial cells and the blood-brain barrier. Third, post-injury administration of an isothiocyanate decreases the loss of tight junction proteins. Fourth, early administration of an isothiocyanate can decrease brain swelling at 24 hr and 3 days following the primary injury. Fifth, delayed administration of an isothiocyanate is highly effective in decreasing brain swelling measured at 3 day after injury. Sixth, administration of an isothiocyanate decreases brain infarct volume following ischemia.

Further investigation also revealed that (a) systemic administration of SUL increases the mRNA level of aquaporin-4 (AQP4) in the brain; and (b) a single administration of SUL 6hr post-injury improves cognitive functions including working memory, memory extinction and spatial memory. These findings suggest a potential therapeutic value for SUL in the treatment of conditions associated with cognitive dysfunction and memory decline.

Therefore, the ability of SUL to reduce brain damage following insults to the CNS could result from protection of brain vasculature, reduction of brain edema, decreasing cell death and improving neuronal function. Thus, SUL may offer two advantages as a therapeutic agent: 1) SUL can induce several enzymes involved in cellular defense, allowing it to act at multiple pathological targets; and 2) since it has been reported that the induction of these genes is long-lasting, this may reduce the necessity for multiple drug administrations.

Sulforaphane, and the related compounds allyl isothiocyanate and phenylethyl isothiocyanate are considered to be representative of their metabolites and of other related or derivative isothiocyanate compounds that have similar physiological or pharmacologic effects to those described herein for sulforaphane.

The results of the present investigations and the foregoing conclusions suggest treatment methods comprising the administration of an isothiocyanate or a related compound to decrease blood-brain barrier compromise, brain swelling, cell loss or cognitive dysfunction following insults to the central nervous system. Other expected benefits from administration of such isothiocyanate compounds to a person in need thereof include: reduced blood-brain barrier break-down and decreased brain swelling or intracranial pressure (ICP) following insults to the central nervous system; lessening of cognitive or motor deficits following brain trauma, stroke or diseases of the central nervous system or aging; decreased central nervous system tissue loss following injury including brain trauma, stroke or spinal cord injury; decreased intracranial pressure or cell loss following infection of the central nervous system; decreased nervous system cell loss as a result of disease and aging; and overcoming toxicity associated with synthetic agents in mammals. An additional advantage of sulforaphane, and other isothiocyanates, is that they are naturally derived compounds.

Cognitive deficits can manifest in the absence of overt neuronal cell loss. For instance, following mild to moderate brain trauma in humans, cognitive and behavioral deficits are observed which can persist for years without observable neurodegeneration²⁰. Moreover, recent studies have shown that although normal aging is not associated with robust neurodegeneration, it is accompanied by measurable cognitive decline^{31,46}. These cognitive deficits are thought to arise from neuronal dysfunction rather than neuronal death. The results presented herein involving the improvement of working memory function in injured animals was observed in the absence of any quantifiable neuronal loss within the prefrontal cortex (data not shown). Taken together, these findings indicate that isothiocyanates can be used to reduce cognitive decline in conditions such as aging, where no demonstrable neuronal loss occurs.

Without further elaboration, it is believed that one skilled in the art can, using the description herein, utilize the present invention to its fullest extent. The foregoing embodiments are to be construed as illustrative, and not as constraining the remainder of the disclosure in any way whatsoever. While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. The disclosures of all patents, patent applications and publications cited herein are hereby incorporated herein by reference, to the extent that they provide exemplary, procedural or other details supplementary to those set forth herein.

### REFERENCES CITED

1. Aronowski J, Strong R, Shirzadi A, Grotta J C. Ethanol plus caffeine (caffeinol) for treatment of ischemic stroke: preclinical experience. Stroke 34:1246-51, 2003.
2. Butt A M, Jones H C and Abbott N J. Electrical resistance across the blood brain barrier in anaesthetized rats: a developmental study. J Physiol. 429:47-62, 1990.
3. Chan P. H., Reactive oxygen radicals in signaling and damage in the ischemic brain, J.Cereb.Blood Flow Metab. 21:2-14, 2001.
4. Chiu D, Krieger D, Villar-Cordova C, Kasner S E, Morgenstern L B, Bratina P L, Yatsu F M and Grotta J C. Intravenous tissue plasminogen activator for acute ischemic stroke: feasibility, safety, and efficacy in the first year of clinical practice. Stroke. 29:18-22, 1998.
5. Clifton G L, Miller E R, Choi S C, Levin H S, McCauley S, Smith K R Jr, Muizelaar J P, Marion D W and Luerssen T G. Hypothermia on admission in patients with severe brain injury. 19:293-301, 2002.
6. Dash P K, Moore A N and Dixon C E. Spatial memory deficits, increased phosphorylation of the transcription factor CREB, and induction of the AP-1 complex following experimental brain injury. J Neuroscience. 15:2030-2039, 1995.
7. Dixon C E, Clifton G L, Lighthall J W, Yaghmai A A, Hayes R L. A controlled cortical impact model of traumatic brain injury in the rat. J Neurosci Methods 39:253-262, 1991
8. Graham D I, Adams J H, Nicoll J A, Maxwell W L, Gennarelli T A. The nature, distribution and causes of traumatic brain injury. Brain Pathol 5:397-406, 1995.
9. Hall E D The neuroprotective pharmacology of methylprednisolone. J Neurosurg.76: 13-22, 1992.
10. Hall E D. Brain attack. Acute therapeutic interventions. Free radical scavengers and antioxidants. Neurosurg Clin N Am. 8:195-206, 1997.
11. Hamann G. F., Y.Okada, R.Fitridge, and G. J.del Zoppo, Microvascular basal lamina antigens disappear during cerebral ischemia and reperfusion, Stroke 26: 2120-2126, 1995.
12. Hu R., V.Hebbar, B. R.Kim, C.Chen, B.Winnik, B.Buckley, P.Soteropoulos, P.Tolias, R. P.Hart, and A. N.Kong, In vivo pharmacokinetics and regulation of gene expression profiles by isothiocyanate sulforaphane in the rat, J.Pharmacol.Exp.Ther. 310: 263-271, 2004.
13. Klatzo I. Presidental address. Neuropathological aspects of brain edema. J Neuropathol Exp Neurol 26:1-14, 1967.
14. Kraft A. D., D. A.Johnson, and J. A.Johnson, Nuclear factor E2-related factor 2-dependent antioxidant response element activation by tert-butylhydroquinone and sulforaphane occurring preferentially in astrocytes conditions neurons against oxidative insult, J.Neurosci. 24:1101-1112, 2004.
15. Lee J. M. and J. A.Johnson, An important role of Nrf2-ARE pathway in the cellular defense mechanism, J.Biochem.Mol.Biol. 37:139-143, 2004.
16. Lee J. M., A. Y.Shih, T. H.Murphy, and J. A.Johnson, NF-E2-related factor-2 mediates neuroprotection against mitochondrial complex I inhibitors and increased concentrations of intracellular calcium in primary cortical neurons, J.Biol.Chem. 278:37948-37956, 2003.
17. Levin H S. Neuroplasticity following non-penetrating traumatic brain injury. Brain Inj. 17:665-74, 2003
18. Levin, H. S. & Hanten, G. Executive functions after traumatic brain injury in children. Pediatr Neurol 33:79-93,2005.
19. Lo E. H., T.Dalkara, and M. A.Moskowitz, Mechanisms, challenges and opportunities in stroke, Nat.Rev.Neurosci. 4:399-415, 2003.
20. Lyeth B G, Jenkins L W, Hamm R J, Dixon C E, Phillips L L, Clifton G L, Young H F, Hayes R L. Prolonged memory impairment in the absence of hippocampal cell death following traumatic brain injury in the rat. Brain Res. 526:249-58, 1990.
21. Manley G T, Binder D K, Papadopoulos M C, Verkman A S. New insights into water transport and edema in the central nervous system from phenotype analysis of aquaporin-4 null mice. Neuroscience 129:983-991, 2004.
22. Manley G T, Fujimura M, Ma T, Noshita N, Filiz F, Bollen A W, Chan P, Verkman A S. Aquaporin-4 deletion in mice reduces brain edema after acute water intoxication and ischemic stroke. Nat Med 6:159-163, 2000.
23. Marmarou A. Traumatic brain edema: an overview. Acta Neurochir Suppl (Wien) 60:421-424, 1994.
24. McIntosh T K, Soares H, Thomas M, Cloherty K. Development of regional cerebral oedema after lateral fluid-percussion brain injury in the rat. Acta Neurochir Suppl (Wien ) 51:263-264, 1990.
25. McWalter G. K., L. G.Higgins, L. I.McLellan, C. J.Henderson, L.Song, P. J.Thomalley, K.Itoh, M.Yamamoto, and J. D.Hayes, Transcription factor Nrf2 is essential for induction of NAD(P)H:quinone oxidoreductase 1, glutathione S-transferases, and glutamate cysteine ligase by broccoli seeds and isothiocyanates, J.Nutr. 134:3499S-3506S, 2004.
26. Meaney D F, Ross D T, Winkelstein B A, Brasko J, Goldstein D, Bilston L B, Thibault L E, Gennarelli T A. Modification of the cortical impact model to produce axonal injury in the rat cerebral cortex. J Neurotrauma 11: 599-612, 1994.
27. Mennicke W H, Gorber K, Krumbiegel G, Lorenz D and Rittmann N. Studies on the metabolism and excretion of benzyl isothiocyanate in man. Xenobiotica 18:441-447, 1988.
28. Nielsen S, Nagelhus E A, Amiry-Moghaddam M, Bourque C, Agre P, Ottersen O P. Specialized membrane domains for water transport in glial cells: high-resolution immunogold cytochemistry of aquaporin-4 in rat brain. J Neurosci 17:171-180, 1997.
29. Papadopoulos M C, Manley G T, Krishna S, Verkman A S. Aquaporin-4 facilitates reabsorption of excess fluid in vasogenic brain edema. FASEB J 18:1291-1293, 2004.
30. Povlishock J. T. and Kontos H. A. Continuing axonal and vascular changes after experimental brain trauma. Cent Nerv Sys Trauma 2:285-298, 1985.
31. Rapp P R, Gallagher M. Preserved neuron number in the hippocampus of aged rats with spatial learning deficits. Proc Natl Acad Sci 93:9926-9930, 1996.
32. Rash J E, Yasumura T, Hudson C S, Agre P, Nielsen S. Direct immunogold labeling of aquaporin-4 in square arrays of astrocyte and ependymocyte plasma membranes in rat brain and spinal cord. Proc Natl Acad Sci U S A 95:11981-11986, 1998.
33. Redell J. B., A. N.Moore, and P. K.Dash, Expression of the prodynorphin gene after experimental brain injury and its role in behavioral dysfunction, Exp.Biol.Med.(Maywood.) 228:261-269, 2003.
34. Rose J., Valtonen S. and Bennet B. Avoidable factors contributing to death after head injury. Brit. Med. J 2:615-618, 1977.
35. Rubin L L, Hall D E, Porter S, Barbu K, Cannon C, Horner H C, Janatpour M, Liaw C W, Manning K, Morales J, et al. A cell culture model of blood brain barrier. J Cell Biol 115:1725-35, 1991.
36. Schulze C, Smales C, Rubin L L and Staddon J M. Lysophosphatidic acid increases tight junction permeability in cultured brain endothelial cells. 68:991-1000, 1997.
37. Shapira Y, Setton D, Artru A A and Shohami E. Blood brain barrier permeability, cerebral edema, and neurological function after closed head injury in rats. Anesth Analg 77: 141-8, 1993.
38. Sharp F. R., A.Lu, Y.Tang, and D. E.Millhorn, Multiple molecular penumbras after focal cerebral ischemia, J.Cereb.Blood Flow Metab 20:1011-1032, 2000.
39. Shohami E, Novikov M, Mechoulam R. A nonpsychotropic cannabinoid, HU-21 1, has cerebroprotective effects after closed head injury in the rat. J Neurotrauma 10: 109-119, 1993.
40. Singh A. V., D.Xiao, K. L.Lew, R.Dhir, and S. V.Singh, Sulforaphane induces caspase-mediated apoptosis in cultured PC-3 human prostate cancer cells and retards growth of PC-3 xenografts in vivo, Carcinogenesis 25:83-90, 2004.
41. Strong R., J. C.Grotta, and J.Aronowski, Combination of low dose ethanol and caffeine protects brain from damage produced by focal ischemia in rats, Neuropharmacology 39:515-522, 2000.
42. Taniguchi M, Yamashita T, Kumura E, Tamatani M, Kobayashi A, Yokawa T, Maruno M, Kato A, Ohnishi T, Kohmura E, Tohyama M, Yoshimine T. Induction of aquaporin-4 water channel mRNA after focal cerebral ischemia in rat. Brain Res Mol Brain Res 78:131-137, 2000.
43. Tanito M., H.Masutani, Y. C.Kim, M.Nishikawa, A.Ohira, and J.Yodoi, Sulforaphane induces thioredoxin through the antioxidant-responsive element and attenuates retinal light damage in mice, Invest Ophthalmol.Vis.Sci. 46:979-987, 2005.
44. Thimmulappa R. K., K. H.Mai, S.Srisuma, T. W.Kensler, M.Yamamoto, and S.Biswal, Identification of Nrf2-regulated genes induced by the chemopreventive agent sulforaphane by oligonucleotide microarray, Cancer Res. 62:5196-5203, 2002.
45. Vermeulen M., van Rooijen H. J. M. and Vaes W., H. J., Analysis of isothiocyanates mercapturic acids in urine: a biomarker for cruciferous vegetable intake. J Agric. Food Chem., 51:3554-3559, 2003.
46. Wickelgren, I., Is hippocampal cell death a myth? Science 271:1229-1230, 1996b.
47. Young W, Bracken MB. The Second National Acute Spinal Cord Injury Study. J Neurotrauma. 1992 Mar;9 Suppl 1:S397-405.
48. Zhang Y., P.Talalay, C. G.Cho, and G. H.Posner, A major inducer of anticarcinogenic protective enzymes from broccoli: isolation and elucidation of structure, Proc.Natl.Acad.Sci.U.S.A 89:2399-2403, 1992.

What is claimed is:

1. A method of treating a mammal suffering from an insult or damage to the central nervous system, or a portion thereof, the method comprising:
administering to the mammal a pharmaceutically effective amount of at least one isothiocyanate, or a derivative or metabolite thereof, to provide at least one of the following results in the mammal:
deterring blood-brain barrier breakdown;
deterring loss of brain tight junction proteins;
deterring central nervous system cell death;
deterring brain swelling and/or intracranial pressure;
reducing brain infarct volume;
improving cognitive function;
thereby lessening or preventing detrimental effects of said insult to the central nervous system or portion thereof.

2. The method of claim 1 wherein said administering further provides at least one of the following effects:
deterring behavioral dysfunctions in said mammal;
deterring cognitive dysfunctions in said mammal,
thereby further lessening detrimental effects of said insult to the central nervous system or portion thereof.

3. The method of claim 1 wherein said administering step comprises administering an effective amount of an isothiocyanate, or a derivative or metabolite thereof, to lessen motor deficits in said mammal arising from said insult.

4. The method of claim 1 wherein said administering step comprises administering an effective amount of an isothiocyanate, or a derivative or metabolite thereof, to decrease loss of central nervous system tissue following said insult.

5. The method of claim 1 wherein said insult comprises trauma, stroke or disease.

6. The method of claim 5 wherein said trauma comprises traumatic brain injury.

7. The method of claim 5 wherein said trauma comprises spinal cord injury.

8. The method of claim 5 wherein said insult comprises stroke, and said administering comprises administering to said mammal an effective amount of at least one isothiocyanate, or a derivative or metabolite thereof, to decrease cell loss in the brain of the mammal.

9. The method of claim 5 wherein said disease comprises an infection of the central nervous system and said administering step comprises administering an effective amount of an isothiocyanate, or a derivative or metabolite thereof, to decrease intracranial pressure and/or cell loss following said infection.

10. The method of claim 1 wherein said administering step comprises administering a natural product comprising an isothiocyanate, or a metabolite thereof, to said mammal.

11. The method of claim 1 wherein said administering step comprises administering an amount of an isothiocyanate, or a derivative or metabolite thereof, sufficient to overcome toxicity associated with a synthetic agent, wherein overcoming said toxicity decreases or prevents death of central nervous system cells in said mammal after exposure to said synthetic agent as a result of said insult to the central nervous system.

12. The method of claim 1 wherein at least one isothiocyanate is chosen from the group consisting of sulforaphane, allyl isothiocyanate and phenylethyl isothiocyanate.

13. A method of attenuating Aquaporin-4 loss in injured brain tissue, the method comprising administering to an mammal suffering from a brain injury an effective amount of an isothiocyanate, or a derivative or metabolite thereof, to enhance or preserve the levels of Aquaporin-4 in at least one area of injured brain tissue in said mammal.

14. A method of attenuating working memory defects in a mammal, comprising: administering a pharmaceutically effective amount of an isothiocyanate, or a derivative or metabolite thereof, to a mammal suffering from a working memory deficit, to improve working memory function in said mammal.

15. The method of claim 14 wherein said administering comprises administering a pharmaceutically effective amount of an isothiocyanate, or a derivative or metabolite thereof, to a mammal suffering from an age-related working memory deficit or to a brain-damaged mammal, thereby improving working memory function in said mammal.

16. A method of improving memory extinction in a mammal, comprising: administering a pharmaceutically effective amount of an isothiocyanate or a derivative or metabolite thereof, to an mammal suffering from an impaired ability to extinguish memory of a traumatic event, to improve memory extinction in said mammal.

17. The method of claim 16 wherein said administering comprises administering said pharmaceutically effective amount of an isothiocyanate, or a derivative or metabolite thereof, to a mammal suffering from age-related impairment of ability to extinguish memory, or to a brain-damaged mammal suffering from an impaired ability to extinguish memory of a traumatic event arising due to brain damage, thereby improving memory extinction in said mammal.

18. A method of improving memory in a mammal in need thereof, comprising: administering a pharmaceutically effective amount of an isothiocyanate, or a derivative or metabolite thereof, to a mammal suffering from memory impairment or deficiency, thereby improving memory in said mammal.

19. The method of claim 18 wherein said administering comprises administering a pharmaceutically effective amount of an isothiocyanate, or a derivative or metabolite thereof, to a mammal suffering from age-related impairment of declarative or spatial memory, or to a brain-damaged mammal suffering from impaired declarative or spatial memory, thereby improving memory in said mammal.


SEQUENCE LISTING (4 sequences)

SEQ ID NO: 1 - DNA, 21 bp, Artificial
  Synthetic
ccagctgtgattccaaacgga

SEQ ID NO: 2 - DNA, 21 bp, Artificial
  Synthetic
gcacagcgcctatgattggtc

SEQ ID NO: 3 - DNA, 19 bp, Artificial
  Synthetic
ccccattgaacacggcatt

SEQ ID NO: 4 - DNA, 22 bp, Artificial
  Synthetic
catcttttcacggttggcctta